(19) United States
(12) Patent Application Publication
(10) Pub. No.: US 2025/0340943 A1
(43) Pub. Date: Nov. 6, 2025

(54) METHODS OF IDENTIFYING CANCERS HAVING A BIALLELIC LOSS OF FUNCTION MUTATION

(71) Applicant: Repare Therapeutics Inc., St-Laurent (CA)

(72) Inventors: Victoria RIMKUNAS, Reading, MA (US); Dominik GLODZIK, Boston, MA (US); Jorge Sergio REIS, New York, NY (US); Robert DABER, Saint-Laurent (CA)

(21) Appl. No.: 18/854,213

(22) PCT Filed: Apr. 6, 2023
(86) PCT No.: PCT/CA2023/050474

§ 371 Date: Oct. 4, 2024

Related U.S. Application Data
(60) Provisional application No. 63/328,499, filed on Apr. 7, 2022.

(51) Int. Cl. C12Q 1/6886; G16B 5/20

(52) U.S. Cl. CPC C12Q 1/6886; G16B 5/20

(57) ABSTRACT

Methods of identifying cancers having a biallelic loss of function mutation (e.g., a STAG2, SETD2, CDK12, ATRIP, REV3L, RAD17, CHTF8, FZR1, RAD51B, RAD51C, RAD51D, PALB2, RNASEH2A, or RNASEH2B loss of function mutation) are disclosed.

### FIELD OF THE INVENTION

The invention relates to methods of identifying a mutation as being biallelic or monoallelic, as well as being germline or somatic.

### BACKGROUND

ATR has been identified as an important cancer target since it is essential for dividing cells. ATR deficient mice are embryonic lethal, however, adult mice with conditional ATR knocked out are viable with effects on rapidly proliferating tissues and stem cell populations. Mouse embryonic stem cells lacking ATR will only divide for 1-2 doublings and then die. Interestingly, mice harboring hypomorphic ATR mutations that reduce expression of ATR to 10% of normal levels showed reduced H-rasG12D-induced tumor growth with minimal effects on proliferating normal cells, e.g., the bone marrow or intestinal epithelial cells.

There is a need for new anti-cancer therapeutic methods and, in particular, those targeting patient populations particularly susceptible to an anti-cancer therapy.

### SUMMARY OF THE INVENTION

In general, the invention provides a method of identifying a cell from a subject as having a biallelic mutation in a target gene, the method including the step of:
from read counts for a plurality of single nucleotide variants (SNVs) (e.g., consistently covered SNVs) including homozygous and heterozygous SNVs (e.g., homozygous and heterozygous consistently covered SNVs) obtained from sequencing a sample including the cell and from reference read counts, determining an integer total copy number of a locus segment within a target gene region in the cell from the subject and/or two integer allele-specific copy numbers of the locus segment, the target gene region including the mutation, where the reference read counts are from a reference population of normal subjects,
where the cell is identified as having a biallelic mutation for a target gene,
if at least one of the integer total copy number and the integer allele-specific copy numbers is 0, provided that the remaining target gene allele, if present, includes the mutation, or
if none of the integer allele-specific copy numbers is 0 and target gene alleles are present, each of the targe gene alleles independently having the mutation.

In some embodiments, the determining step includes the steps of:
from the read counts (e.g., read counts for an alternative allele) and the reference read counts (e.g., read counts for the reference allele), determining total copy number log-ratios, allelic copy number log-odds ratios, and target coverage values for the SNVs;
segmenting the total copy number log-ratios and the allelic copy number log-odds ratios;
estimating sample purity and sample ploidy for the cell from the total copy number log-ratios and the target coverage values; and
from the target coverage values, the sample purity, the sample ploidy, the total copy number log-ratios, and the allelic copy number log-odds ratios, generating an integer total copy number of a segment including a plurality of SNVs within a target gene region in the cell and two integer allele-specific copy numbers of the segment.

In some embodiments, the method further includes the step of adjusting the ratios for location shift.

In a further aspect, the invention provides a method of identifying a target mutation in a cell from a subject as being germline or somatic, the method including the steps of:
from read counts for a plurality of consistently covered single nucleotide variants (SNVs) including homozygous and heterozygous consistently covered SNVs obtained from sequencing a sample including the cell and from reference read counts, determining an observed allele fraction of a locus segment within a target gene region in the cell from the subject, the target gene region including the target mutation;
determining expected allele fractions for a germline target mutation and for a somatic target mutation;
comparing the observed allele fraction to the expected allele fractions to identify the most probable of the germline and somatic mutations; and
identifying the target mutation as germline or somatic as that which is the most probable for the germline and somatic mutations.

In yet further aspect, the invention provides a method of identifying a target mutation in a cell from a subject as being germline or somatic, the method including identifying the target mutation in the normal, matched sample from the subject,
where
if the target mutation present in the cell from the subject is identified in the normal, matched sample, the target mutation is germline, and
if the target mutation present in the cell from the subject is not identified in the normal, matched sample, the target mutation is somatic.

In some embodiments of any of the aspects, the comparing step is performed using Bayesian model comparison. In some embodiments of any of the aspects, each of the consistently covered SNVs has the mean coverage of at least 200× reads across panel of normal samples. In some embodiments of any of the aspects, the plurality of SNVs includes SNVs with an allele frequency of 33% to 66% in humans. In some embodiments, the plurality of SNVs includes SNVs proximal to the frequent SNVs (e.g., disposed within 300 contiguous nucleobases downstream from the frequent SNV). In some embodiments, the plurality of SNVs includes SNVs, each of the SNVs having a 5′-flanking sequence of at least 20 contiguous nucleobases including 25-75% GC content, where the 5′-flanking sequence is unique and does not include other SNVs. In some embodiments of any of the aspects, the plurality of SNVs includes at least 20 heterozygous SNVs. In some embodiments of any of the aspects, the plurality of SNVs includes scaffold SNVs (e.g., scaffold SNVs may be useful to limit the solution space for the integer total copy number and integer allele-specific copy numbers). In some embodiments of any of the aspects, the target gene region includes the target gene and flanking regions up to 10 kilobases each. In some embodiments of any of the aspects, the target gene region includes the target gene and flanking regions up to 5 kilobases each. In some embodiments of any of the aspects, the target gene region includes the target gene and flanking regions up to 2 kilobases each. In some embodiments of any of the aspects, the target gene region is a target exome region. In some embodiments of any of the aspects, the target gene region is a target transcriptome region. In some embodiments of any of the aspects, the target gene region is a target genome region. In some embodiments of any of the aspects, the cell from the subject is a cancer cell from the subject.

In some embodiments of any of the aspects, the target is STAG2. In some embodiments of any of the aspects, the target is SETD2. In some embodiments of any of the aspects, the target is CDK12. In some embodiments of any of the aspects, the target is ATRIP. In some embodiments of any of the aspects, the target is REV3L. In some embodiments of any of the aspects, the target is RAD17. In some embodiments of any of the aspects, the target is CHTF8. In some embodiments of any of the aspects, the target is FZR1. In some embodiments of any of the aspects, the target is RAD51B. In some embodiments of any of the aspects, the target is RAD51C. In some embodiments of any of the aspects, the target is RAD51D. In some embodiments of any of the aspects, the target is PALB2. In some embodiments of any of the aspects, the target is RNASEH2A. In some embodiments of any of the aspects, the target is RNASEH2B.

In some embodiments of any of the aspects, the mutation is a germline mutation.

### Definitions

The term “allele fraction,” as used herein, refers to a normalized measure of the allelic intensity ratio of a variant allele, such that an allele fraction of 1 or 0 indicates the complete absence of one of the two alleles. For ploidy of 2, an allele fraction of 0.5 indicates the equal presence of both alleles. For ploidy of 3, an allele fraction of 0.33 or 0.66 indicates the presence of one copy of one allele and two copies of another allele. For ploidy of 4, an allele fraction of 0.25 or 0.75 indicates the presence of one copy of one allele and three copies of another allele, and an allele fraction of 0.5 indicates the equal presence of both alleles. An allele fraction can be measured as a B Allele Frequency.

The term “allelic copy number log-odds ratio,” as used herein, refers to a ratio of parental copy numbers in a cancer cell (E[log OR]=[p1·Φ+(1−Φ)]/[p2·Φ+(1−Φ)]), where E[log OR] is the expected value of log OR, p1 is a parental copy number of the variant allele, p2 is a parental copy number of the allele from the other parent, and Φ is a cellular fraction that is a function of tumor purity and clonal frequency (for subclonal alterations).

The term “biallelic loss of function mutation,” as used herein, refers to a mutation within a subject's cell (e.g., cancer cell) that results in the elimination of the active form of a target gene in the cell. For example, a “biallelic STAG2, SETD2, CDK12, ATRIP, REV3L, RAD17, CHTF8, FZR1, RAD51B, RAD51C, RAD51D, PALB2, RNASEH2A, or RNASEH2B loss of function mutation” refers to a mutation within a subject's cell (e.g., cancer cell) that results in the elimination of the active form of a STAG2, SETD2, CDK12, ATRIP, REV3L, RAD17, CHTF8, FZR1, RAD51B, RAD51C, RAD51D, PALB2, RNASEH2A, or RNASEH2B gene in the cell.

The term “BRCA2,” as used herein, represents a breast cancer type 2 susceptibility gene or protein.

The term “cancer,” as used herein, refers to all types of cancer, neoplasm or malignant tumors found in mammals (e.g., humans), including leukemia, carcinomas and sarcomas. Non-limiting examples of cancers that may be treated with a compound or method provided herein include prostate cancer, thyroid cancer, endocrine system cancer, brain cancer, breast cancer, cervix cancer, colon cancer, head & neck cancer, liver cancer, kidney cancer, lung cancer, non-small cell lung cancer, melanoma, mesothelioma, ovarian cancer, sarcoma, stomach cancer, uterus cancer, medulloblastoma, ampullary cancer, colorectal cancer, and pancreatic cancer. Additional non-limiting examples may include, Hodgkin's disease, Non-Hodgkin's lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphoma, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, and prostate cancer.

The term “carcinoma,” as used herein, refers to a malignant new growth made up of epithelial cells tending to infiltrate the surrounding tissues and give rise to metastases. Non-limiting examples of carcinomas that may be treated with a compound or method provided herein include, e.g., medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

“Disease” or “condition” refer to a state of being or health status of a patient or subject capable of being treated with the compounds or methods provided herein.

The term “gene region” or “target gene region” is a nucleotide region within a genome that partly or wholly includes a target gene (e.g., a stromal antigen 2 (STAG2), a SET domain containing 2 (SETD2), a cyclin-dependent kinase 12 (CDK12), an ATR interacting protein (ATRIP), a reversionless 3-like (REV3L), a RAD17, a chromosome transmission fidelity factor 8 (CHTF8), a fizzy and cell division cycle 20 related 1(FZR1), a RAD51B, a RAD51C, a RAD51D, a partner and localizer of BRCA2 (PALB2), a ribonuclease H2 subunit A (RNASEH2A), or a ribonuclease H2 subunit B (RNASEH2B)).

The term “leukemia,” as used herein, refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, e.g., acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “lymphoma,” as used herein, refers to a cancer arising from cells of immune origin. Non-limiting examples of T and B cell lymphomas include non-Hodgkin lymphoma and Hodgkin disease, diffuse large B-cell lymphoma, follicular lymphoma, mucosa-associated lymphatic tissue (MALT) lymphoma, small cell lymphocytic lymphoma-chronic lymphocytic leukemia, Mantle cell lymphoma, mediastinal (thymic) large B-cell lymphoma, lymphoplasmacytic lymphoma-Waldenstrom macroglobulinemia, peripheral T-cell lymphoma (PTCL), angioimmunoblastic T-cell lymphoma (AITL)/follicular T-cell lymphoma (FTCL), anaplastic large cell lymphoma (ALCL), enteropathy-associated T-cell lymphoma (EATL), adult T-cell leukaemia/lymphoma (ATLL), or extranodal NK/T-cell lymphoma, nasal type.

The term “melanoma,” as used herein, is taken to mean a tumor arising from the melanocytic system of the skin and other organs. Melanomas that may be treated with a compound or method provided herein include, e.g., acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma.

The term “Next Generation Sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

The term “RNAse H2A,” as used herein, refers to Ribonuclease H2, subunit A.

The term “RNAse H2B,” as used herein, refers to Ribonuclease H2, subunit B.

The term “sarcoma” generally refers to a tumor which is made up of a substance like the embryonic connective tissue and is generally composed of closely packed cells embedded in a fibrillar or homogeneous substance. Non-limiting examples of sarcomas that may be treated with a compound or method provided herein include, e.g., a chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “scaffold SNV,” as used herein, represent frequent, well-covered single nucleotide variants outside the target gene region and spaced throughout the chromosome carrying the target gene region.

The term “STAG2”, as used herein, refers to Stromal Antigen 2.

The term “subject,” as used herein, represents a human or non-human animal (e.g., a mammal) that is suffering from, or is at risk of, disease or condition, as determined by a qualified professional (e.g., a doctor or a nurse practitioner) with or without known in the art laboratory test(s) of sample(s) from the subject. Preferably, the subject is a human. Non-limiting examples of diseases and conditions include diseases having the symptom of cell hyperproliferation, e.g., a cancer.

The term “target” or “target gene” refers to one or more of the following genes: stromal antigen 2 (STAG2), SET domain containing 2 (SETD2), cyclin-dependent kinase 12 (CDK12), an ATR interacting protein (ATRIP), reversionless 3-like (REV3L), RAD17, chromosome transmission fidelity factor 8 (CHTF8), fizzy and cell division cycle 20 related 1(FZR1), RAD51B, RAD51C, RAD51 D, partner and localizer of BRCA2 (PALB2), ribonuclease H2 subunit (RNASEH2A), and ribonuclease H2 subunit B (RNASEH2B)).

The term “target coverage,” as used herein, refers to the average number of reads aligning to a chromosomal position in a target gene region.

The term “total copy number log-ratio,” as used herein, refers to a cancer cell over control cell signal ratio. The total copy number log-ratio deviations from an average of 0 for a given region suggest signal intensity to be higher (if greater than 0) or lower (if less than 0) than expected for two chromosomal copies. The total copy number log-ratio, also known as Log R, may be estimated using GenomeStudio® software from Illumina.

“Treatment” and “treating,” as used herein, refer to the medical management of a subject with the intent to improve, ameliorate, stabilize, prevent or cure a disease or condition. This term includes active treatment (treatment directed to improve the disease or condition); causal treatment (treatment directed to the cause of the associated disease or condition); palliative treatment (treatment designed for the relief of symptoms of the disease or condition); preventative treatment (treatment directed to minimizing or partially or completely inhibiting the development of the associated disease or condition); and supportive treatment (treatment employed to supplement another therapy). A disease or condition may be a cancer.

### BRIEF DESCRIPTION OF THE DRAWINGS

FIGS. 1A-1C illustrates three mechanisms of germline loss of function mutations in a target (e.g., STAG2, SETD2, CDK12, ATRIP, REV3L, RAD17, CHTF8, FZR1, RAD51B, RAD51C, RAD51D, PALB2, RNASEH2A, or RNASEH2B). FIGS. 1A and 1C illustrate monoallelic germline loss of function mutations, and FIG. 1B illustrates a biallelic germline loss of function mutation.

FIG. 2A is a chart showing the copy number calls across all chromosomes as determined by whole genome sequencing (WGS) for a female subject, age 46, with papillary serous carcinoma.

FIG. 2B is a chart showing the copy number calls across all chromosomes as determined using a single nucleotide variant (SNV) Panel Version 1 for a female subject, age 46, with papillary serous carcinoma.

FIG. 3A is a chart showing the copy number calls across all chromosomes as determined by WGS for a female subject, age 62, with pancreatic ductal adenocarcinoma.

FIG. 3B is a chart showing the copy number calls across all chromosomes as determined using a SNV Panel Version 1 for a female subject, age 62, with pancreatic ductal adenocarcinoma.

FIG. 4A is a chart showing the copy number calls across all chromosomes as determined by WGS for a female subject, age 72, with metastatic breast carcinoma (ER−, PR−, Her2−).

FIG. 4B is a chart showing the copy number calls across all chromosomes as determined using a SNV Panel Version 1 for a female subject, age 72, with metastatic breast carcinoma (ER−, PR−, Her2−).

FIG. 5A is a chart showing the single nucleotide variant (SNV) coverage of SNV Panel Version 1 on chromosome 1.

FIG. 5B is a chart showing the SNV coverage of SNV Panel Version 2 on chromosome 1.

FIG. 6 is a series of charts showing the sequencing coverage depth (top graph), b-allele fraction (middle graph), and copy number profile (bottom graph) for a cancer biopsy from a subject.

FIG. 7A is a chart showing the copy number calls across all chromosomes as determined by WGS for a subject, age 55, with lung adenocarcinoma.

FIG. 7B is a chart showing the copy number calls across all chromosomes as determined using a SNV Panel Version 2 for a subject, age 55, with lung adenocarcinoma.

FIG. 8A is a chart showing the copy number calls across all chromosomes as determined by WGS for a subject, age 72, with breast cancer, lum B.

FIG. 8B is a chart showing the copy number calls across all chromosomes as determined using a SNV Panel Version 2 for a subject, age 72, with breast cancer, lum B.

FIG. 9A is a chart showing the copy number calls across all chromosomes as determined by WGS for a subject, age 41, with bladder cancer.

FIG. 9B is a chart showing the copy number calls across all chromosomes as determined using a SNV Panel Version 2 for a subject, age 41, with bladder cancer.

FIG. 10A is a chart showing the copy number calls across all chromosomes as determined by WGS for a subject, age 80, with bladder cancer.

FIG. 10B is a chart showing the copy number calls across all chromosomes as determined using a SNV Panel Version 2 for a subject, age 80, with bladder cancer.

FIG. 11A is a chart showing the copy number calls across all chromosomes as determined by WGS for a subject, age 66, with breast luminal B cancer.

FIG. 11B is a chart showing the copy number calls across all chromosomes as determined using a SNV Panel Version 2 for a subject, age 66, with breast luminal B cancer.

FIG. 12A is a chart showing the copy number calls across all chromosomes as determined by WGS for a subject, age 62, with prostate cancer.

FIG. 12B is a chart showing the copy number calls across all chromosomes as determined using a SNV Panel Version 2 for a subject, age 62, with prostate cancer.

FIG. 13A is a chart showing the copy number calls across all chromosomes as determined by WGS for a subject, age 69, with uterus cancer.

FIG. 13B is a chart showing the copy number calls across all chromosomes as determined using a SNV Panel Version 2 for a subject, age 69, with uterus cancer.

FIG. 14A is a chart showing the copy number calls across all chromosomes as determined by WGS for a subject, age 46, with triple-negative breast cancer.

FIG. 14B is a chart showing the copy number calls across all chromosomes as determined using an SNV Panel Version 2 for a subject, age 46, with triple-negative breast cancer.

FIG. 15 is a plot showing the sequencing coverage (x reads) observed for a series of downstream positions downstream from the primer binding site. This chart shows that better quality samples produce higher sequencing coverage over all positions. For the panel of normal samples, only good and fair samples were used.

### DETAILED DESCRIPTION

The methods of the invention address a problem of distinguishing a biallelic loss-of-function mutation from a monoallelic loss-of-function mutation as well as distinguishing germline and somatic mutations. Advantageously, the methods of the invention expressly account for sample purity and therefore are substantially unaffected by contaminated samples. A further advantage of the methods of the invention is in that, they can utilize pre-existing data from a panel of normal samples (normal non-cancerous tissue from a reference population) and do not require a normal tissue sample from the subject.

Typically, the subjects have a monoallelic germline loss of function mutation and subsequently acquire a somatic loss of function mutation for the same gene (e.g., STAG2, SETD2, CDK12, ATRIP, REV3L, RAD17, CHTF8, FZR1, RAD51B, RAD51C, RAD51D, PALB2, RNASEH2A, or RNASEH2B). These subjects thus have a biallelic loss of function mutation.

### Identification of Biallelic Loss of Function

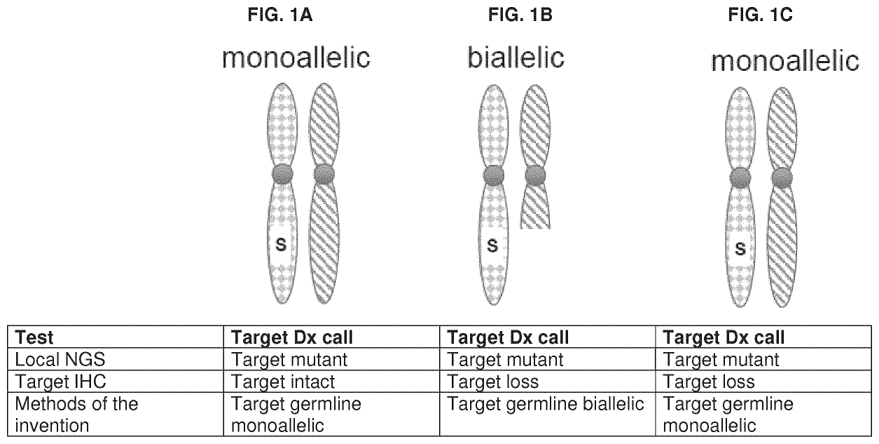

A subject or a cancer cell therefrom may be identified as having a biallelic loss of function for a gene using, e.g., Whole Genome Sequencing (WGS) or Whole Exome Sequencing (WES). Methods of the invention address the need for identification of biallelic loss of function mutation. Three exemplary mechanisms of loss of function mutations are illustrated in FIGS. 1A-1C. FIGS. 1A and 1C illustrate monoallelic loss of function mutations, and FIG. 1B illustrates a biallelic loss of function mutation. Typical next generation sequencing techniques used in cancer tests fail to distinguish between these mechanisms. Immunohistochemistry (IHC) fails to distinguish between the biallelic mutation in FIG. 1B and the monoallelic mutation in FIG. 1C, which results in an apparent target (e.g., STAG2, SETD2, CDK12, ATRIP, REV3L, RAD17, CHTF8, FZR1, RAD51B, RAD51C, RAD51D, PALB2, RNASEH2A, or RNASEH2B) protein loss. As described herein, the methods involving identification of the biallelic loss of function mutations distinguish the biallelic mutation in FIG. 1B from the monoallelic mutations in FIGS. 1A and 1C.

Advantageously, methods presented herein identify a subject or a cancer cell therefrom as having a biallelic loss of function for a gene but with greater cost efficiency and target gene coverage than WGS and WES techniques.

Typically, a method of the invention may include a step of determining from read counts for a plurality of single nucleotide variants (SNVs) including homozygous and heterozygous SNVs obtained from sequencing a sample including the cancer cell and from reference read counts, determining an integer total copy number of a locus segment within a target gene (e.g., STAG2, SETD2, CDK12, ATRIP, REV3L, RAD17, CHTF8, FZR1, RAD51B, RAD51C, RAD51D, PALB2, RNASEH2A, or RNASEH2B) region in a cancer cell from the subject or in the cancer cell and/or two integer allele-specific copy numbers of the locus segment, wherein the cancer is identified as having a biallelic (e.g., STAG2, SETD2, CDK12, ATRIP, REV3L, RAD17, CHTF8, FZR1, RAD51B, RAD51C, RAD51 D, PALB2, RNASEH2A, or RNASEH2B) loss of function mutation if at least one of the integer total copy number and the integer allele-specific copy numbers is 0. When the integer total copy number is 0, the detected mutation is a homozygous deletion. Thus, the homozygous deletion would indicate a biallelic loss-of-function mutation for the target gene (e.g., STAG2, SETD2, CDK12, ATRIP, REV3L, RAD17, CHTF8, FZR1, RAD51B, RAD51C, RAD51D, PALB2, RNASEH2A, or RNASEH2B). When the integer total copy number is >0, and the integer allele-specified copy number is 0 (e.g., at the locus where the target-inactivating mutation is found), the detected mutation is a loss-of-heterozygosity. Thus, if the remaining target gene (e.g., STAG2, SETD2, CDK12, ATRIP, REV3L, RAD17, CHTF8, FZR1, RAD51B, RAD51C, RAD51D, PALB2, RNASEH2A, or RNASEH2B) allele comprises an inactivating mutation, the integer allele-specified copy number of 0 would indicate that the subject has a biallelic loss-of-function mutation for the target gene (e.g., STAG2, SETD2, CDK12, ATRIP, REV3L, RAD17, CHTF8, FZR1, RAD51B, RAD51C, RAD51D, PALB2, RNASEH2A, or RNASEH2B). For example, the step of determining may include: from read counts for the plurality of SNVs including homozygous and heterozygous SNVs obtained from sequencing a sample comprising the cancer cell and from reference read counts, determining total copy number log-ratios, allelic copy number log-odds ratios, and target coverage values for the heterozygous SNVs; segmenting the total copy number log-ratios and the allelic copy number log-odds ratios; estimating sample purity and sample ploidy for the cancer cell from the total copy number log-ratios and the target coverage values; and from the target coverage values, the sample purity, the sample ploidy, the total copy number log-ratios, and the allelic copy number log-odds ratios, generating an integer total copy number of a segment comprising a plurality of heterozygous single nucleotide variants (SNVs) within a target gene region (e.g., STAG2, SETD2, CDK12, ATRIP, REV3L, RAD17, CHTF8, FZR1, RAD51B, RAD51C, RAD51D, PALB2, RNASEH2A, or RNASEH2B gene region) in the cancer cell and two integer allele-specific copy numbers of the segment. Typically, the cell from the subject is provided as a biopsy. Read counts may be obtained using next generation sequencing of the cells in the sample.

Alternatively, the method of the invention may utilize B allele frequency analysis to identify biallelic (e.g., STAG2, SETD2, CDK12, ATRIP, REV3L, RAD17, CHTF8, FZR1, RAD51B, RAD51C, RAD51 D, PALB2, RNASEH2A, or RNASEH2B) loss of function. For example, this method may include: determining a plurality of allele fractions for SNVs within a target gene region (e.g., STAG2, SETD2, CDK12, ATRIP, REV3L, RAD17, CHTF8, FZR1, RAD51B, RAD51C, RAD51 D, PALB2, RNASEH2A, or RNASEH2B gene region) in a cancer cell from the subject or in the cancer cell, and segmenting the plurality of allele fractions to produce a plurality of constant allele fraction segments, wherein the cancer is identified as having a biallelic loss of function mutation (e.g., biallelic STAG2, SETD2, CDK12, ATRIP, REV3L, RAD17, CHTF8, FZR1, RAD51B, RAD51C, RAD51D, PALB2, RNASEH2A, or RNASEH2B loss of function mutation) if the target gene region (e.g., STAG2, SETD2, CDK12, ATRIP, REV3L, RAD17, CHTF8, FZR1, RAD51B, RAD51C, RAD51D, PALB2, RNASEH2A, or RNASEH2B gene region) comprises a locus of SNVs lacking segments with allele fractions between 0.05 and 0.95.

Among the methods described herein, the methods utilizing integer allele-specific copy numbers and integer total copy numbers are advantageous over others, as these methods are robust and could be used to process low purity samples. Additionally, the methods described herein and utilizing integer allele-specific copy numbers and integer total copy numbers can utilize pre-existing data from a panel of normal samples from a reference population and do not require a normal tissue sample from the subject. Thus, such a method allows for determination of a biallelic loss-of-function mutation based on a single sample (e.g., a biopsy) form the subject.

### Target SNVs

Target SNVs to be used in the methods of the invention can be selected from those known in the art according to several selection criteria identified below. The SNVs can be found, e.g., at gnomad.broadinstitute.org.

A target SNV is preferably consistently covered across samples. A target SNV is consistently covered across samples, if its mean coverage is at least 50× reads (e.g., at least 100× reads, at least 200× reads, at least 300× reads, at least 400× reads, or at least 500× read,) across the panel of normal samples. The panel of target SNVs may have a mean coverage of at least 50× (e.g., at least 100×, at least 200×, at least 300×, at least 400×, at least 500×, at least 600×, at least 700×, at least 800×, at least 900×, or at least 1000× (e.g., 100× to 2500×, 200× to 2500×, 300× to 2500×, 400× to 2500×, 500× to 2500×, 600× to 2500×, 700× to 2500×, 800× to 2500×, 900× to 2500× or 1000× to 2500×)) across the panel of normal samples. Panel of normal samples are derived from normal tissue of the reference population, where chromosomes are expected to be normal. Panel of normal samples has SNV allele fractions of 0 to 0.1 for homozygous variants, 0.4 to 0.6 for heterozygous variants, and 0.9 to 1 for absent variants. Typically, the panel of normal samples is assembled from the samples of the same tissue type as those from the subject's sample.

A target SNV may be a frequent SNV, for example, the frequent SNV may be that which has an allele frequency of greater than 33% (e.g., 33% to 66%) in humans. Here, the assessment of allele frequency in humans may be based on an SNV source, e.g., Gnomad. The inbreeding coefficient for the reference population may be between 0 and 0.2. Additionally, a target SNV may be a proximal SNV—a consistently covered SNV that is disposed within a 3′-flanking sequence relative to the frequent SNV, the 3′-flanking sequence including at total of 300 contiguous nucleobases.

A target SNV may have a 5′-flanking sequence of at least 20 contiguous nucleobases (e.g., 20-50 contiguous nucleobases, e.g., 50 contiguous nucleobases) including 25-75% GC content. Typically, the 5′-flanking sequence is unique (i.e., the sequence of 20 contiguous nucleobases is not found elsewhere within the target genome) and does not include other SNVs.

A target SNV may be a clean SNV. A clean SNV has the variant allele fraction (VAF) values within ranges 0-0.1, 0.4-0.6, and 0.9-1 in at least 95% of samples from the reference population.

Typically, target SNVs may be detected using primer-based detection techniques (e.g., next generation sequencing techniques). For a plurality of target SNVs, a plurality of primers may be designed using techniques and methods known in the art. When selecting target SNVs from a sequenced sample containing a cancer cell from a subject, those target SNVs may be selected that are disposed within the 3′-flanking sequences relative to the binding sites for the utilized plurality of primers. The 3′-flanking sequence is typically a sequence containing 300 or fewer (e.g., 200 or fewer) contiguous nucleobases in the 3′ direction relative to the binding site for the utilized primer. The number of contiguous nucleobases selected for a 3′-flanking sequence may be affected by the level of DNA damage, and length of DNA fragments in each patient sample. For example, for the mean coverage of 100× or more (e.g., 200× or more), the 3′-flanking sequence of 200 or fewer contiguous nucleobases may be used. For example, the 3′-flanking sequence of 300 bp for samples with >17% of input DNA fragments longer than 130 bp, and the 3′-flanking sequence of 200 bp otherwise. As a general matter, the 3′-flanking sequence length may be adjusted in view of the sequencing technology utilized in the sample analysis and the sample quality, the lower quality samples (i.e., samples with high degree of DNA fragmentation) typically necessitate the use of shorter 3′-flanking sequences and/or higher mean coverage levels.

Advantageously, the method described herein does not require the subject's normal tissue sample to determine whether a mutation is monoallelic or biallelic. Instead, the method described herein may utilize reference population samples. For example, reads from the panel of normal samples may be used instead of normal reads in the BAM files.

### Total Copy Number Log-Ratio

A total copy number log-ratio (Log R) may be generated from the total read count in the cancer versus reference for all target SNVs that have at least a minimum depth of coverage in the reference. Log R provides information on total copy number ratio. Sequence read count information may be first parsed from paired cancer-reference files. A normalizing constant is calculated for each cancer/reference pair to correct for total library size. Subsampling within 150-250 bp intervals may be applied to reduce hypersegmentation in SNV-dense regions of the genome. Specifically, the expected value of Log R can be expressed as

$$E [ log R ] = log \{ ( p {1}^{*} + p {2}^{*} ) / 2 \} + w ( \cdot ) + λ ,$$

where p1*=p1·Φ+(1−Φ) and p2*=p2·Φ+(1−Φ) are parental copy number in the tumor sample rising from a mixed normal (1,1) and aberrant (p1,p2) copy number genotype with mixing proportion Φ. Φ is the cellular fraction associated with the aberrant genotype, which is a function of tumor purity and clonal frequency (for subclonal alterations). The term w(·) denotes systematic bias. GC-content may be explicitly considered, and loess regression of log R over GC in 1 kb windows along the genome may be used to estimate the GC-effect on read counts and subtract it from log R. In addition, Log R quantifies relative copy number, hence a constant A is included for absolute copy number conversion.

For Log R generation, sequence read count information may be first parsed form paired cancer-control BAM files. A normalizing constant may be calculated for each cancer/control pair to correct for total library size. Subsampling within 150-250 bp intervals may be applied to reduce hypersegmentation in SNV-dense regions of the genome.

### Allelic Copy Number Log-Odds Ratio

Allelic copy number log-odds ratio (log OR) of the variant-allele count in cancer versus reference allele, which is an unbiased estimate of allelic copy ratio: E[log OR]=[p1·Φ+(1−Φ)]/[p2·Φ+(1−Φ)]), where E[log OR] is the expected value of log OR, p1 is a parental copy number of the variant allele, p2 is a parental copy number of the allele from the other parent, and Φ is a cellular fraction that is a function of tumor purity and clonal frequency (for subclonal alterations). In the absence of phased data, squared log OR may be used to infer log²([p1·Φ+(1−Φ)]/[p2·Φ+(1−Φ)]).

### Joint Segmentation

Segmentation analysis may be used to identify regions of the genome that have constant copy number using change point detection methods. One preferred method is described in Shen et al., Nucleic Acids Res., 2016, 44(16): e131; doi: 10.1093/nar/gkw520, hereby incorporated by reference. In this method, a circular binary segmentation algorithm is used for a joint segmentation of log R and log OR based on a bivariate Hotelling T² statistic:

$${T}^{2} = {max}_{1 \leq i \leq j \leq n} {T}_{1 ij}^{2} + {cT}_{2 ij}^{2}$$

where T₁ᵢⱼ the Mann-Whitney statistic comparing the set of observed log R denoted as {X₁ₖ: i<k≤j} and its complement {X₁ₖ: 1<k≤i or j<k≤n}, and T₂ᵢⱼ is the Mann-Whitney statistic comparing the set of observed log OR denoted as {X₂ₖ: i<k≤j} and its complement {X₂ₖ: 1<k≤i or j<k≤n} (Shen et al., Nucleic Acids Res., 2016, 44(16): e131; doi: 10. 1093/nar/gkw520). In the above, c is a scaling factor that is inversely proportional to the heterozygous rate.

If the maximal statistic is greater than a pre-determined critical value, a change is declared and the change-points are estimated as i, j that maximize the statistic. This approach iteratively searches for change points between any possible pair of breakpoints and its complement to identify regions of the genome that have constant allele-specific copy number. For each segment, the log R data are summarized using the median of the log R values [symbol] and the log OR data are summarized by

$${\tilde{x}}_{2}^{2}$$

which takes the form Σ{x₂²−s²)/s²}/Σ{1/s²}, where s² is the estimated variance of log OR.

While log R is defined for all SNVs (both homozygous and heterozygous loci), log OR is only defined for heterozygous loci (het-loci or het-SNVs). This might create an imbalance between the two in the combined statistic. To address this issue, a weight that is inversely proportional to the heterozygous rate is introduced to increase the het-SNV contributions in subsequent segmentation analysis. Specifically, a scaling factor c is introduced in the T² statistic. This is empirically set at 1/\sqrt{4γ}, where γ is the proportion of het-SNVs in the cancer cell sample. Up-weighing the contribution of log OR for het-SNVs increases the power of detecting allelic imbalances for regions with low frequency of het-SNVs.

Segmentation may be alternatively performed using, for example, a running mean method. Alternatively, the Log R and Log OR data may be divided into predetermined short segments (based on the SNV loci).

After segmentation, the segments are clustered into groups of the same underlying genotype. Such clustering reduces the number of latent copy number and cellular fraction states needed in subsequent modeling.

### Location Shift Determination

Log R estimates are proportional to the absolute total copy number up to a location constant λ. For diploid genome, log R=0 (library size normalized log R) is the location for the 2-copy state. However, aneuploidy can lead to a location shift in the tumor. Therefore, the 2-copy state should be determined in a tumor genome, and the location constant λ should be quantified.

The copy number states may be denoted using total and minor integer copy number (e.g. 1-0 denotes monosomy with total copy number 1 and minor copy number 0). The estimate of λ should correspond to the log R level at which the segments are in 2-1 (normal diploid) or 2-0 (copy-neutral LOH) state. In order to estimate λ, normal diploid segments should be allelically balanced. Thus, candidate value for λ (referred to as λ_{c}) will be obtained from [symbol] for segment clusters that have [symbol] values close to zero.

However, homozygous deletions (0-0) and balanced gains (4-2, 6-3 etc.) are also allelically balanced and hence will have small [symbol] . Since large scale homozygous deletions of multiple genes will not be conducive to cell survival, non-focal segments with small [symbol] may be eliminated as being homozygous deletions. In addition, for the sake of simplicity, higher order balanced gains states (6-3, 8-4 etc.) spanning a large part of the genome are not considered. Samples in which segments with allelic balance are a small fraction of targeted regions are flagged and may be subjected to a manual review for their λ estimates.

In samples that have large allelically balanced segments, there can be several [symbol] values from which λc can be chosen.

### Integer Copy Numbers

An integer allele-specific copy numbers (major and minor) and the associated cellular fraction estimates for each segment cluster by modeling the expected values of log R and log OR given total (t), and each parental (p1,p2) copy as a function of a cf parameter Φ, using a combination of parametric and non-parametric methods. This allows for modeling both clonal and subclonal events.

First a moment estimate of [symbol] , the total copy number for segment cluster i, is obtained by

$$\vert {2}^{( 1 - \text{?}} ,$$

$$\text{?} \text{indicates text missing or illegible when filed}$$

where [symbol] denote the median log R for segment cluster i corrected for sequence bias and tumor ploidy (λ-normalized. Once the total number is obtained, we calculate the allele specific copy numbers m and p and the cellular fraction Φ using the fact that the log OR summary measure [symbol] is a moment estimate of μ² which equals log

$${2}^{} ( \{ p 1 \cdot Φ + ( 1 - Φ ) \} / \{ p 2 \cdot Φ + ( 1 - Φ ) \} ) .$$

To further refine the initial estimates, a Gaussian-non-central χ² model may employed with error terms to account for the noise with a clonal structure imposed on the cellular fraction Φ. Specifically, let X₁ᵢⱼ denote the log R for SNV loci j in segment cluster i (corrected for sequence bias and location shift) and follow a normal distribution:

$${X}_{1 ij} ∼ N ( {v}_{ig} , {{T}_{i}}^{2} ) ,$$

where v_{ig} is the expected value of log R given the underlying copy number state g taking the form

$${v}_{ig} = {log}_{2} ( 2 ( 1 - {ϕ}_{k} ) + {t}_{g} {ϕ}_{k} ) / 2 ,$$

where t_{g}=p1_{g}+p2_{g} denotes the total copy number (sum of the two parental copy number) given the underlying copy number state g, Φₖ denotes the cellular fraction for clonal cluster k, and τᵢ² is an independent variance parameter. In practice, it is reasonable to assume homoscedasticity and set τᵢ²=τ²∀i.

Furthermore, let X₂ᵢⱼ denote the log OR for SNV loci j in segment cluster i and (X₂ᵢⱼ/σᵢⱼ)² follow a non-central chi-squared distribution:

$${( {X}_{2 ij} / {σ}_{ij} )}^{2} ∼ {X}^{2} ( {δ}_{ijg} ) ,$$

where σᵢⱼ² is the variance parameter for log OR and δ_{ijg}=μᵢⱼ²/σᵢⱼ² is the non-centrality parameter in which

$${{μ}_{ig}}^{2} = {log}_{2} ( ( {m}_{g} {ϕ}_{k} + ( 1 - {ϕ}_{k} ) ) / ( {p}_{g} {ϕ}_{k} + ( 1 - {ϕ}_{k} ) ) ) .$$

Assuming X₁ᵢⱼ and X₂ᵢⱼ are independent random variables given the underlying copy number state g, the joint data likelihood can then be written as

$$ℓ = {\sum }_{i} {\sum }_{j} {\sum }_{g} f ( {x}_{1 ij} \vert {v}_{ig} , {τ}_{i}^{2} , g ) f ( {x}_{2 ij} \vert {δ}_{ijg} , g ) P ( g )$$

where P(g) is the prior probability of the latent copy number state g.

An expectation-maximization (EM) algorithm may be applied to improve the joint data likelihood. It can be viewed as an estimation problem with the latent copy number states as missing data. In the E-step of the EM procedure, Bayes theorem is used to compute the posterior probability of segment cluster i being assigned copy number state g given the parameter estimates at the tth iteration:

$${\hat{p}}_{ijg}^{( t )} = \frac{f ( {x}_{1 ij} \vert {\hat{v}}_{ig}^{( t )} , {\hat{τ}}_{i}^{{2}^{( t )}} , g ) f ( {x}_{2 ij} \vert {\hat{δ}}_{ijg}^{( t )} ) P ( g )}{{\sum }_{g} f ( {x}_{1 ij} \vert {\hat{v}}_{ig} , {\hat{τ}}_{i}^{{2}^{( t )}} , g ) f ( {x}_{2 ij} \vert {\hat{δ}}_{ijg}^{( t )} ) P ( g )} .$$

In the M-step, we first update the normal and non-central Chi-square distribution parameters

$${\hat{v}}_{ig}^{( t + 1 )} = \frac{{\sum }_{j} {\hat{p}}_{ijg}^{( t )} \cdot {x}_{1 ij}}{{\sum }_{j} {\hat{p}}_{ijg}^{( t )}} \cdot {\hat{τ}}_{i}^{{2}^{( t )}} = \frac{{\sum }_{j} {{\hat{p}}_{ijg}^{( t )} ( {x}_{1 ij} - {\hat{v}}_{ig}^{( t )} )}^{2}}{{\sum }_{j} {\hat{p}}_{ijg}^{( t )}} .$$

$${\hat{μ}}_{ig}^{{2}^{( t + 1 )}} = \frac{{\sum }_{j} {\hat{p}}_{ijg}^{( t )} \cdot ( {x}_{2 ij}^{2} - {s}^{2} ) / {s}^{2}}{{\sum }_{j} {\hat{p}}_{ijg}^{( t )} / {s}^{2}} .$$

where s² is the sample variance estimate of log OR. After obtaining the estimates of v and then update the cellular fraction parameter ϕₖ^{(t+1)} given

$$\text{?} = log \frac{2 ( 1 - {ϕ}_{k} ) + \text{?} {ϕ}_{k}}{2} \cdot \text{?} = log \frac{\text{?} {ϕ}_{i} - ( 1 - {ϕ}_{k} )}{\text{?} {ϕ}_{k} + ( 1 - {ϕ}_{k} )} .$$

$$\text{?} \text{indicates text missing or illegible when filed}$$

where g* is the most likely genotype (with highest posterior probability) given the data and current parameter estimates in the tth iteration. The E-step and M-step are iterated until convergence.

A clonal structure is imposed on the cellular fraction Φₖ. This is done in a sequential approach where the algorithm starts with a single clonal cluster (k=1) with cellular fraction parameter Φ₁. Then, the method may involve identification of segment clusters for which segment cluster-specific estimates are non-trivially lower (at least by 0.05) from the clonally constrained estimates that result in a suboptimal fit under k=1. These segment clusters with discordant cellular fraction estimates then form a candidate subclonal cluster of events at a lower cellular fraction Φ₂, and a model is fitted with the joint likelihood optimized under k=2. This procedure is iterated until no additional discordance in cellular fraction estimates are found, or a specified maximum k (e.g., k=5) is reached, as desired and depending on the intratumor heterogeneity. In the output, [symbol] is the cellar fraction estimate for the clonal events and also the tumor purity by definition, and [symbol] , k>1 for any subclonal clusters identified in the sample.

### Distinguishing Germline and Somatic Mutations

The methods described herein may be used to identify a target mutation as germline or somatic.

Using methods described herein, identification of the target mutation as a germline or somatic mutation may be achieved with or without the use of a normal, matched sample from the subject (in addition to the sample containing a cancer cell from the subject, e.g., a biopsy). A normal, matched sample from the same subject is a sample containing normal (non-cancerous) cells, e.g., a blood sample from the subject.

In instances where a sample containing a cancer cell from the subject (e.g., a biopsy from the subject) and a normal, matched sample from the subject (e.g., a blood sample from the subject) are available, the methods described herein may include the step of identifying a mutation in the normal, matched sample from the subject. If the target mutation present in the cancer cell from the subject is identified in the normal, matched sample, the target mutation is germline. If the target mutation present in the cancer cell from the subject is not identified in the normal, matched sample, the target mutation is somatic.

For example, in instances where a normal, matched sample from the subject is unavailable, the methods described herein may include the steps of:
from read counts for a plurality of consistently covered single nucleotide variants (SNVs) comprising homozygous and heterozygous consistently covered SNVs obtained from sequencing a sample comprising the cell, determining an observed allele fraction of the target mutation,
determining expected allele fractions (E[a]) for a germline mutation and for a somatic mutation, where the allele fraction for the germline mutation is E[a]=[(1−Φ)·1+Φ·m_{cn}]/[(1−Φ)·2+Φ·tₜᵤₘ], and m_{cn} is p1 or p2, tₜᵤₘ is total copy number of the locus in tumor, and where the allele fractions for the somatic mutation are E[a]=[Φ·m_{cn}]/[(1−Φ)·2+Φ·tₜᵤₘ] enumerated for all m_{cn}≤min(p1,p2), where m_{cn} is a mutant allele copy number for the genome region including the target mutation, Φ is a cellular fraction (used as a measure of the sample purity), p1 is a parental copy number of the variant allele, p2 is a parental copy number of the allele from the other parent,
comparing the observed allele fraction to the expected allele fractions to identify the most probable of the germline and somatic mutations, and
identifying the target mutation as germline or somatic as that which is the most probable of the germline and somatic mutations.

The mutant allele copy number (m_{cn}) is an integer from 1 to tₜᵤₘ, where tₜᵤₘ is the total copy number of alleles for the region of interest in the cancer cell from the subject. The comparing step may be performed using Bayesian model comparison (Bayes factor).

This approach presumes that the normal cells are diploid and that the sample from the subject is impure (Φ<1). As Φ approaches 1, the germline and somatic mutations become indistinguishable in this approach in the absence of the normal, matched sample from the subject.

Alternatively, e.g., in instances where a normal, matched sample from the subject is unavailable, the sample containing a cancer cell from the subject is impure (Φ<0.9, or 90%), and there is an absence of somatic copy number changes, the methods described herein may be used to identify a target mutation as somatic if the observed allele fraction is outside the expected range of allele fraction (unadjusted for purity). For example, for a total copy number of 2, SNVs would be expected to occur within an allele fraction range (unadjusted for purity) of less than 10% (homozygous SNV that is absent), 40-60% (e.g., 45-55%) (heterozygous SNV), and greater than 90% (homozygous SNV that is present); therefore, the target mutation is somatic, if its observed allele fraction is outside the expected allele fraction ranges. If the observed allele fraction is within an expected allele fraction range, this particular approach does not permit characterizing the target mutation.

### Biallelic Target Loss of Function Mutation Identification

Methods described herein may include a step of identifying the cancer having as a biallelic STAG2, SETD2, CDK12, ATRIP, REV3L, RAD17, CHTF8, FZR1, RAD51B, RAD51C, RAD51D, PALB2, RNASEH2A, or RNASEH2B loss of function mutation using the techniques described above.

### SNV Detection

Detection techniques for evaluating nucleic acids for the presence of a SNV involve procedures well known in the field of molecular genetics. Many, but not all, of the methods involve amplification of nucleic acids. Ample guidance for performing amplification is provided in the art. Exemplary references include manuals such as PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Current Protocols in Molecular Biology, Ausubel, 1994-1999, including supplemental updates through April 2004; Sambrook & Russell, Molecular Cloning, A Laboratory Manual (3rd Ed, 2001). General methods for detection of single nucleotide variants are disclosed in Single Nucleotide Variants: Methods and Protocols, Pui-Yan Kwok, ed., 2003, Humana Press. SNV detection methods often employ labeled oligonucleotides. Oligonucleotides can be labeled by incorporating a label detectable by spectroscopic, photochemical, biochemical, immunochemical, or chemical means. Useful labels include fluorescent dyes, radioactive labels, e.g. 32P, electron-dense reagents, enzyme, such as peroxidase or alkaline phosphatase, biotin, or haptens and proteins for which antisera or monoclonal antibodies are available. Labeling techniques are well known in the art (see, e.g. Current Protocols in Molecular Biology, supra; Sambrook & Russell, supra).

Although the methods typically employ PCR steps, other amplification protocols may also be used. Suitable amplification methods include ligase chain reaction (see, e.g., Wu & Wallace, Genomics 4:560-569, 1988); strand displacement assay (see, e.g. Walker et al., Proc. Natl. Acad. Sci. USA 89:392-396, 1992; U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., Proc. Natl. Acad. Sci. USA 86:1173-1177, 1989); and self-sustained sequence replication (3SR) (Guatelli et al., Proc. Natl. Acad. Sci. USA 87:1874-1878, 1990; WO 92/08800). Alternatively, methods that amplify the probe to detectable levels can be used, such as Qβ-replicase amplification (Kramer & Lizardi, Nature 339:401-402, 1989; Lomeli et al., Clin. Chem. 35:1826-1831, 1989). A review of known amplification methods is provided, for example, by Abramson and Myers in Curr. Op Biotechnol. 4:41-47, 1993.

Detection of the genotype, haplotype, SNV, microsatellite, or other variant of an individual can be performed using oligonucleotide primers and/or probes. Oligonucleotides can be prepared by any suitable method, usually chemical synthesis. Oligonucleotides can be synthesized using commercially available reagents and instruments. Alternatively, they can be purchased through commercial sources. Methods of synthesizing oligonucleotides are well known in the art (see, e.g., Narang et al., Meth. Enzymol. 68:90-99, 1979; Brown et al., Meth. Enzymol. 68:109-151, 1979; Beaucage et al., Tetrahedron Lett. 22:1859-1862, 1981; and the solid support method of U.S. Pat. No. 4,458,066). In addition, modifications to the above-described methods of synthesis may be used to desirably impact enzyme behavior with respect to the synthesized oligonucleotides. For example, incorporation of modified phosphodiester linkages (e.g., phosphorothioate, methylphosphonates, phosphoamidate, or boranophosphate) or linkages other than a phosphorous acid derivative into an oligonucleotide may be used to prevent cleavage at a selected site. In addition, the use of 2′-amino modified sugars tends to favor displacement over digestion of the oligonucleotide when hybridized to a nucleic acid that is also the template for synthesis of a new nucleic acid strand.

The genotype of an individual can be determined using many detection methods that are well known in the art. Most assays entail one of several general protocols: hybridization using allele-specific oligonucleotides, primer extension, allele-specific ligation, sequencing, or electrophoretic separation techniques, e.g., single-stranded conformational variant (SSCP) and heteroduplex analysis. Exemplary assays include 5′-nuclease assays, template-directed dye-terminator incorporation, molecular beacon allele-specific oligonucleotide assays, single-base extension assays, and SNV scoring by real-time pyrophosphate sequences. Analysis of amplified sequences can be performed using various technologies such as microchips, fluorescence polarization assays, and MALDI-TOF (matrix assisted laser desorption ionization-time of flight) mass spectrometry. Two methods that can also be used are assays based on invasive cleavage with Flap nucleases and methodologies employing padlock probes.

Determination of the presence or absence of a particular allele is generally performed by analyzing a nucleic acid sample that is obtained from the individual to be analyzed. Often, the nucleic acid sample comprises genomic DNA. The genomic DNA is typically obtained from blood samples but may also be obtained from other cells or tissues.

It is also possible to analyze RNA samples for the presence of polymorphic alleles. For example, mRNA can be used to determine the genotype of an individual at one or more polymorphic sites. In this case, the nucleic acid sample is obtained from cells in which the target nucleic acid is expressed, e.g., adipocytes. Such an analysis can be performed by first reverse-transcribing the target RNA using, e.g., a viral reverse transcriptase, and then amplifying the resulting cDNA; or using a combined high-temperature reverse-transcription-polymerase chain reaction (RT-PCR), as described in U.S. Pat. Nos. 5,310,652; 5,322,770; 5,561,058; 5,641,864; and 5,693,517.

Frequently used methodologies for analysis of nucleic acid samples to detect SNVs are briefly described. However, any method known in the art can be used in the invention to detect the presence of single nucleotide substitutions.

### Allele-Specific Hybridization

Allele-specific hybridization, also commonly referred to as allele specific oligonucleotide hybridization (ASO) (e.g., Stoneking et al., Am. J. Hum. Genet. 48:70-382, 1991; Saiki et al., Nature 324, 163-166, 1986; EP 235,726; and WO 89/11548), relies on distinguishing between two DNA molecules differing by one base by hybridizing an oligonucleotide probe that is specific for one of the variants to an amplified product obtained from amplifying the nucleic acid sample. This method typically employs short oligonucleotides, e.g. 15-20 bases in length. The probes are designed to differentially hybridize to one variant versus another. Principles and guidance for designing such probe is available in the art, e.g. in the references cited herein. Hybridization conditions should be sufficiently stringent that there is a significant difference in hybridization intensity between alleles, and producing an essentially binary response, whereby a probe hybridizes to only one of the alleles. Some probes are designed to hybridize to a segment of target DNA such that the polymorphic site aligns with a central position (e.g., in a 15-base oligonucleotide at the 7 position; in a 16-based oligonucleotide at either the 8 or 9 position) of the probe, but this design is not required.

The amount and/or presence of an allele is determined by measuring the amount of allele-specific oligonucleotide that is hybridized to the sample. Typically, the oligonucleotide is labeled with a label such as a fluorescent label. For example, an allele-specific oligonucleotide is applied to immobilized oligonucleotides representing SNV sequences. After stringent hybridization and washing conditions, fluorescence intensity is measured for each SNV oligonucleotide.

In one embodiment, the nucleotide present at the polymorphic site is identified by hybridization under sequence-specific hybridization conditions with an oligonucleotide probe or primer exactly complementary to one of the polymorphic alleles in a region encompassing the polymorphic site. The probe or primer hybridizing sequence and sequence-specific hybridization conditions are selected such that a single mismatch at the polymorphic site destabilizes the hybridization duplex sufficiently so that it is effectively not formed. Thus, under sequence-specific hybridization conditions, stable duplexes will form only between the probe or primer and the exactly complementary allelic sequence. Thus, oligonucleotides from about 10 to about 35 nucleotides in length, usually from about 15 to about 35 nucleotides in length, which are exactly complementary to an allele sequence in a region which encompasses the polymorphic site are within the scope of the invention.

In an alternative embodiment, the nucleotide present at the polymorphic site is identified by hybridization under sufficiently stringent hybridization conditions with an oligonucleotide substantially complementary to one of the SNV alleles in a region encompassing the polymorphic site, and exactly complementary to the allele at the polymorphic site. Because mismatches which occur at non-polymorphic sites are mismatches with both allele sequences, the difference in the number of mismatches in a duplex formed with the target allele sequence and in a duplex formed with the corresponding non-target allele sequence is the same as when an oligonucleotide exactly complementary to the target allele sequence is used. In this embodiment, the hybridization conditions are relaxed sufficiently to allow the formation of stable duplexes with the target sequence, while maintaining sufficient stringency to preclude the formation of stable duplexes with non-target sequences. Under such sufficiently stringent hybridization conditions, stable duplexes will form only between the probe or primer and the target allele. Thus, oligonucleotides from about 10 to about 35 nucleotides in length, usually from about 15 to about 35 nucleotides in length, which are substantially complementary to an allele sequence in a region which encompasses the polymorphic site and are exactly complementary to the allele sequence at the polymorphic site, are within the scope of the invention.

The use of substantially, rather than exactly, complementary oligonucleotides may be desirable in assay formats in which optimization of hybridization conditions is limited. For example, in a typical multi-target immobilized-oligonucleotide assay format, probes or primers for each target are immobilized on a single solid support. Hybridizations are carried out simultaneously by contacting the solid support with a solution containing target DNA. As all hybridizations are carried out under identical conditions, the hybridization conditions cannot be separately optimized for each probe or primer. The incorporation of mismatches into a probe or primer can be used to adjust duplex stability when the assay format precludes adjusting the hybridization conditions. The effect of a particular introduced mismatch on duplex stability is well known, and the duplex stability can be routinely both estimated and empirically determined, as described above. Suitable hybridization conditions, which depend on the exact size and sequence of the probe or primer, can be selected empirically using the guidance provided herein and well known in the art. The use of oligonucleotide probes or primers to detect single base pair differences in sequence is described in, e.g., Conner et al., Proc. Natl. Acad. Sci. USA 80:278-282, 1983, and U.S. Pat. Nos. 5,468,613 and 5,604,099, each incorporated herein by reference.

The proportional change in stability between a perfectly matched and a single-base mismatched hybridization duplex depends on the length of the hybridized oligonucleotides. Duplexes formed with shorter probe sequences are destabilized proportionally more by the presence of a mismatch. Oligonucleotides between about 15 and about 35 nucleotides in length are often used for sequence-specific detection. Furthermore, because the ends of a hybridized oligonucleotide undergo continuous random dissociation and re-annealing due to thermal energy, a mismatch at either end destabilizes the hybridization duplex less than a mismatch occurring internally. For discrimination of a single base pair change in target sequence, the probe sequence that hybridizes to the target sequence is selected such that the polymorphic site occurs in the interior region of the probe.

The above criteria for selecting a probe sequence that hybridizes to a specific allele apply to the hybridizing region of the probe, i.e., that part of the probe which is involved in hybridization with the target sequence. A probe may be bound to an additional nucleic acid sequence, such as a poly-T tail used to immobilize the probe, without significantly altering the hybridization characteristics of the probe. One of skill in the art will recognize that for use in the present methods, a probe bound to an additional nucleic acid sequence which is not complementary to the target sequence and, thus, is not involved in the hybridization, is essentially equivalent to the unbound probe.

Suitable assay formats for detecting hybrids formed between probes and target nucleic acid sequences in a sample are known in the art and include the immobilized target (dot-blot) format and immobilized probe (reverse dot-blot or line-blot) assay formats. Dot blot and reverse dot blot assay formats are described in U.S. Pat. Nos. 5,310,893; 5,451,512; 5,468,613; and 5,604,099; each incorporated herein by reference.

In a dot-blot format, amplified target DNA is immobilized on a solid support, such as a nylon membrane. The membrane-target complex is incubated with labeled probe under suitable hybridization conditions, unhybridized probe is removed by washing under suitably stringent conditions, and the membrane is monitored for the presence of bound probe.

In the reverse dot-blot (or line-blot) format, the probes are immobilized on a solid support, such as a nylon membrane or a microtiter plate. The target DNA is labeled, typically during amplification by the incorporation of labeled primers. One or both of the primers can be labeled. The membrane-probe complex is incubated with the labeled amplified target DNA under suitable hybridization conditions, unhybridized target DNA is removed by washing under suitably stringent conditions, and the membrane is monitored for the presence of bound target DNA. A reverse line-blot detection assay is described in the example.

An allele-specific probe that is specific for one of the variant variants is often used in conjunction with the allele-specific probe for the other variant. In some embodiments, the probes are immobilized on a solid support and the target sequence in an individual is analyzed using both probes simultaneously. Examples of nucleic acid arrays are described by WO 95/11995. The same array or a different array can be used for analysis of characterized variants. WO 95/11995 also describes subarrays that are optimized for detection of variant forms of a pre-characterized variant. Such a subarray can be used in detecting the presence of the variants described herein.

### Allele-Specific Primers

Variants are also commonly detected using allele-specific amplification or primer extension methods. These reactions typically involve use of primers that are designed to specifically target a variant via a mismatch at the 3′-end of a primer. The presence of a mismatch effects the ability of a polymerase to extend a primer when the polymerase lacks error-correcting activity. For example, to detect an allele sequence using an allele-specific amplification- or extension-based method, a primer complementary to one allele of a variant is designed such that the 3-terminal nucleotide hybridizes at the polymorphic position. The presence of the particular allele can be determined by the ability of the primer to initiate extension. If the 3-terminus is mismatched, the extension is impeded.

In some embodiments, the primer is used in conjunction with a second primer in an amplification reaction. The second primer hybridizes at a site unrelated to the polymorphic position. Amplification proceeds from the two primers leading to a detectable product signifying the particular allelic form is present. Allele-specific amplification- or extension-based methods are described in, e.g., WO 93/22456; U.S. Pat. Nos. 5,137,806; 5,595,890; 5,639,611; and 4,851,331.

Using allele-specific amplification-based genotyping, identification of the alleles requires only detection of the presence or absence of amplified target sequences. Methods for the detection of amplified target sequences are well known in the art. For example, gel electrophoresis and probe hybridization assays described are often used to detect the presence of nucleic acids.

In an alternative probe-less method, the amplified nucleic acid is detected by monitoring the increase in the total amount of double-stranded DNA in the reaction mixture, is described, e.g. in U.S. Pat. No. 5,994,056; and European Patent Publication Nos. 487,218 and 512,334. The detection of double-stranded target DNA relies on the increased fluorescence various DNA-binding dyes, e.g., SYBR Green, exhibit when bound to double-stranded DNA.

As appreciated by one in the art, allele-specific amplification methods can be performed in reaction that employ multiple allele-specific primers to target particular alleles. Primers for such multiplex applications are generally labeled with distinguishable labels or are selected such that the amplification products produced from the alleles are distinguishable by size. Thus, for example, both alleles in a single sample can be identified using a single amplification by gel analysis of the amplification product.

As in the case of allele-specific probes, an allele-specific oligonucleotide primer may be exactly complementary to one of the polymorphic alleles in the hybridizing region or may have some mismatches at positions other than the 3′-terminus of the oligonucleotide, which mismatches occur at non-polymorphic sites in both allele sequences.

### Detectable Probes

### 5′-Nuclease Assay Probes

Genotyping can also be performed using a “TaqMan®” or “5′-nuclease assay”, e.g., as described in U.S. Pat. Nos. 5,210,015; 5,487,972; and 5,804,375; and Holland et al., Proc. Natl. Acad. Sci. USA 88:7276-72801988. In the TaqMan® assay, labeled detection probes that hybridize within the amplified region are added during the amplification reaction. The probes are modified so as to prevent the probes from acting as primers for DNA synthesis. The amplification is performed using a DNA polymerase having 5′- to 3′-exonuclease activity. During each synthesis step of the amplification, any probe which hybridizes to the target nucleic acid downstream from the primer being extended is degraded by the 5′- to 3′-exonuclease activity of the DNA polymerase. Thus, the synthesis of a new target strand also results in the degradation of a probe, and the accumulation of degradation product provides a measure of the synthesis of target sequences.

The hybridization probe can be an allele-specific probe that discriminates between the SNV alleles. Alternatively, the method can be performed using an allele-specific primer and a labeled probe that binds to amplified product.

Any method suitable for detecting degradation product can be used in a 5′-nuclease assay. Often, the detection probe is labeled with two fluorescent dyes, one of which is capable of quenching the fluorescence of the other dye. The dyes are attached to the probe, usually one attached to the 5′-terminus and the other is attached to an internal site, such that quenching occurs when the probe is in an unhybridized state and such that cleavage of the probe by the 5′- to 3′-exonuclease activity of the DNA polymerase occurs in between the two dyes. Amplification results in cleavage of the probe between the dyes with a concomitant elimination of quenching and an increase in the fluorescence observable from the initially quenched dye. The accumulation of degradation product is monitored by measuring the increase in reaction fluorescence. U.S. Pat. Nos. 5,491,063 and 5,571,673, both incorporated herein by reference, describe alternative methods for detecting the degradation of probe which occurs concomitant with amplification.

### Secondary Structure Probes

Probes detectable upon a secondary structural change are also suitable for detection of a variant, including SNVs. Exemplified secondary structure or stem-loop structure probes include molecular beacons or Scorpion® primer/probes. Molecular beacon probes are single-stranded oligonucleic acid probes that can form a hairpin structure in which a fluorophore and a quencher are usually placed on the opposite ends of the oligonucleotide. At either end of the probe short complementary sequences allow for the formation of an intramolecular stem, which enables the fluorophore and the quencher to come into close proximity. The loop portion of the molecular beacon is complementary to a target nucleic acid of interest. Binding of this probe to its target nucleic acid of interest forms a hybrid that forces the stem apart. This causes a conformation change that moves the fluorophore and the quencher away from each other and leads to a more intense fluorescent signal. Molecular beacon probes are, however, highly sensitive to small sequence variation in the probe target (Tyagi and Kramer, Nat. Biotechnol. Vol. 14, pages 303-308, 1996; Tyagi et al., Nat. Biotech, Vol. 16, pages 49-53, 1998; Piatek et al., Nat Biotechnol, 16:359-363 (1998); Marras et al., Genetic Analysis: Biomolecular Engineering, Vol 14, pages 151-156 (1999); Täpp I. et al, BioTechniques. Vol 28, pages 732-738, 2000). A Scorpion® primer/probe comprises a stem-loop structure probe covalently linked to a primer.

### Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution (see, e.g., Erlich, ed., PCR Technology: Principles and Applications for DNA Amplification, W. H. Freeman and Co, New York, 1992, Chapter 7).

Distinguishing of microsatellite variants can be done using capillary electrophoresis. Capillary electrophoresis conveniently allows identification of the number of repeats in a particular microsatellite allele. The application of capillary electrophoresis to the analysis of DNA variants is well known to those in the art (see, e.g., Szantai, et al, J Chromatogr A. 1079(1-2):41-49, 2005; Bjørheim and Ekstrøm, Electrophoresis 26(13):2520-2530, 2005 and Mitchelson, Mol Biotechnol. 24(1):41 68, 2003).

### Single-Strand Conformation Polymorphism Analysis

Alleles of target sequences can be differentiated using single-strand conformation polymorphism analysis, which identifies base differences by alteration in electrophoretic migration of single stranded PCR products, as described, e.g., in Orita et al., Proc. Natl. Acad. Sci. USA 86(8), 2766-2770, 1989. Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target.

### DNA Sequencing and Single Base Extensions

SNVs can also be detected by direct sequencing. Methods include e.g. dideoxy sequencing-based methods and other methods such as Maxam and Gilbert sequence (see, e.g. Sambrook and Russell, supra).

Other detection methods include Pyrosequencing™ of oligonucleotide-length products. Such methods often employ amplification techniques such as PCR. For example, in pyrosequencing, a sequencing primer is hybridized to a single stranded, PCR-amplified, DNA template; and incubated with the enzymes, DNA polymerase, ATP sulfurylase, luciferase and apyrase, and the substrates, adenosine 5′ phosphosulfate (APS) and luciferin. The first of four deoxynucleotide triphosphates (dNTP) is added to the reaction. DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. ATP sulfurylase quantitatively converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a Pyrogram™. Each light signal is proportional to the number of nucleotides incorporated. Apyrase, a nucleotide degrading enzyme, continuously degrades unincorporated dNTPs and excess ATP. When degradation is complete, another dNTP is added.

Another similar method for characterizing SNVs does not require use of a complete PCR, but typically uses only the extension of a primer by a single, fluorescence-labeled dideoxyribonucleic acid molecule (ddNTP) that is complementary to the nucleotide to be investigated. The nucleotide at the polymorphic site can be identified via detection of a primer that has been extended by one base and is fluorescently labeled (e.g., Kobayashi et al, Mol. Cell. Probes, 9:175-182, 1995).

Additionally, SNVs can be determined from analyses (e.g., computational analyses) of data obtained from next generation sequencing (NGS) experiments Buermans and Dunnen. Biochimica et Biophysica Acta. 1842:1932-1941, 2014). Sequencing can be performed by various systems currently available, such as, without limitation, a sequencing system by ILLUMINA®, Pacific Biosciences (PACBIO®), Oxford NANOPORE®, or Life Technologies (ION TORRENT®). Methods, reagents, and equipment for performing these different sequencing systems can be obtained from their respective manufacturers. Alternatively or additionally, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads. A read may include a string of nucleic acid bases corresponding to a sequence of a nucleic acid molecule that has been sequenced.

SNVs can be identified from data generated by NGS experiments by comparing the occurrence of different nucleic acid base pairs at the same locus across multiple samples. Due to errors that occur in NGS sequencing, probabilistic models (e.g. Bayesian models) are often used to distinguish and correct read errors from true SNVs. A wide variety of methods and algorithms have been developed to detect SNVs from NGS data (see, e.g., Nielsen et al. Nat. Rev. Genet. 12(6):443-451, 2011; Bansal, Bioinformatics. 26(12):i318-i324, 2010; Roth et al. Bioinformatics. 28(7):907-913, 2012; You et al. Bioinformatics. 28(5):643-650, 2012; Li et al., Genome Res. 19(6):1124-1132, 2009; Abecasis et al. Nature. 467(7319):1061-1073, 2010; Larson et al. Bioinformatics. 28(3):311-317, 2012). Resources for identifying SNVs found in the human genome include databases of sequenced genomes (e.g., gnomAD, Bravo, ClinVar, 1000 Genome Project, and TopMed) and databases of identified SNVs (e.g., dbSNP, HapMap, Biomart, SPSmart, and Genome Variation Server (GVS)).

### EXAMPLES

### Example 1. Design of SNV Panel Version 1

An SNV panel based on Anchored Multiplex PCR (AMP™) technology was designed to quantify genetic alterations in the ATM gene. Gene-specific primers were designed to amplify genomic regions of interesting utilizing AMP chemistry.

Gene-specific primers were designed for up to 30 single nucleotide variants (SNVs) that appear in 20-80% of the global population (as defined in the gnomAD database v.2.1.1) for ATM target.

Sample ploidy and chromosomal arm loss were assessed using gene-specific primers designed for 1000 randomly distributed SNVs throughout the genome.

The procedure described above may also be used for the assessment of STAG2, SETD2, CDK12, ATRIP, REV3L, RAD17, CHTF8, FZR1, RAD51B, RAD51C, RAD51D, PALB2, RNASEH2A, or RNASEH2B as a target gene.

### Example 2. Evaluation of SNV Panel Version 1 Performance Against Whole Genome Sequencing

The SNV panel designed according to the process outlined in Example 1 permits quantification of loss of heterozygosity and copy number loss for ATM. Furthermore, the design of the SNV Panel exhibits full exonic coverage, and also provides quantification of genome-wide SNVs for purity and ploidy calls. The performance of the SNV Panel was evaluated against whole genome sequencing (WGS) for 3 samples from subjects having various cancers. In particular, the three subjects were a female, age 46, with papillary serous carcinoma; a female, age 62, with pancreatic ductal adenocarcinoma; and a female, age 72, with metastatic breast carcinoma, ER−, PR−, HER2−.

For preparation of each SNV Panel sample, DNA obtained from the sample was amplified using the SNV Panel primers using VariantPlex® cycling, optimized for large panels of >3500 primer pairs:
i. PCR 1/2 cycle number 10/15
ii. Extension time: 15 min
iii. Anneal/extend temperature 60° C./65° C.
iv. Expanded testing of primer amounts in PCR
   Amplified DNA libraries were sequenced and normalized to 12 million reads per library.

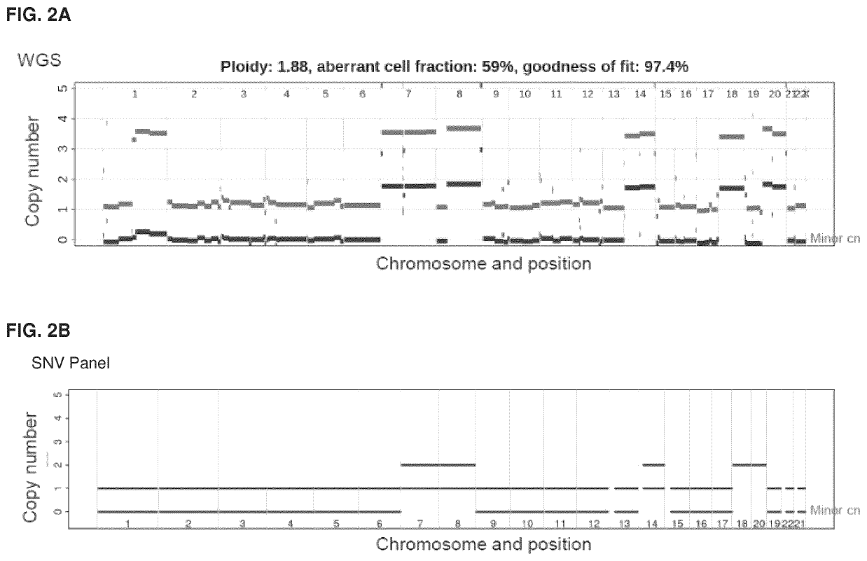

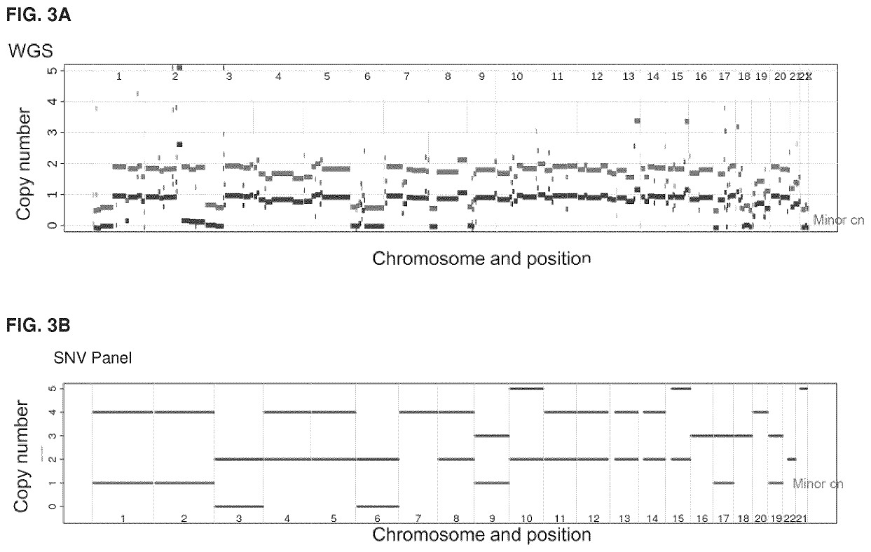

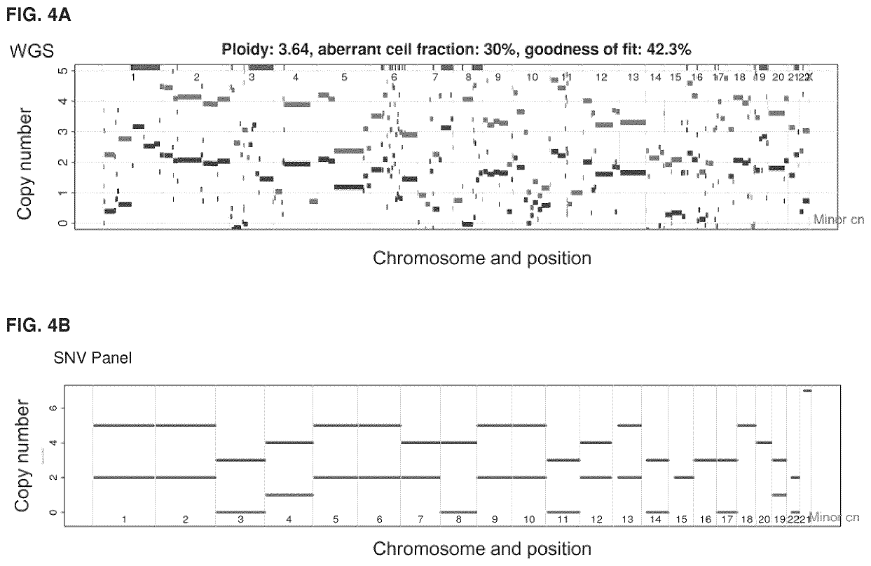

Quantification and comparison of copy number calls across all chromosomes as determined by WGS and the SNV Panel for the 3 exemplary samples are shown in FIGS. 2A, 2B, 3A, 3B, 4A, and 4B. In particular, the ability for the SNV Panel to accurately identify LOH was evaluated, with WGS serving as the basis for comparison. SNV Panel and WGS exhibited good agreement in calling LOH in the 3 exemplary samples (see Tables 2-4), particularly in tumors with simpler karyotypes and high cancer cell fractions in the sample.

TABLE 2
Concordance of LOH calls per gene

WGS

| | | LOH | Heterozygosity |
|---|---|---|---|
| SNV Panel | LOH | 35 | 0 |
| Version 1 | Heterozygosity | 0 | 5 |

TABLE 3
Concordance of LOH calls per gene

WGS

| | | LOH | Heterozygosity |
|---|---|---|---|
| SNV Panel | LOH | 8 | 1 |
| Version 1 | Heterozygosity | 4 | 27 |

TABLE 4
Concordance of LOH calls per gene

WGS

| | | LOH | Heterozygosity |
|---|---|---|---|
| SNV Panel | LOH | 17 | 5 |
| Version 1 | Heterozygosity | 1 | 17 |

The procedure described above may also be used for the assessment of STAG2, SETD2, CDK12, ATRIP, REV3L, RAD17, CHTF8, FZR1, RAD51B, RAD51C, RAD51D, PALB2, RNASEH2A, or RNASEH2B as a target gene.

### Example 3. Design of SNV Panel Version 2

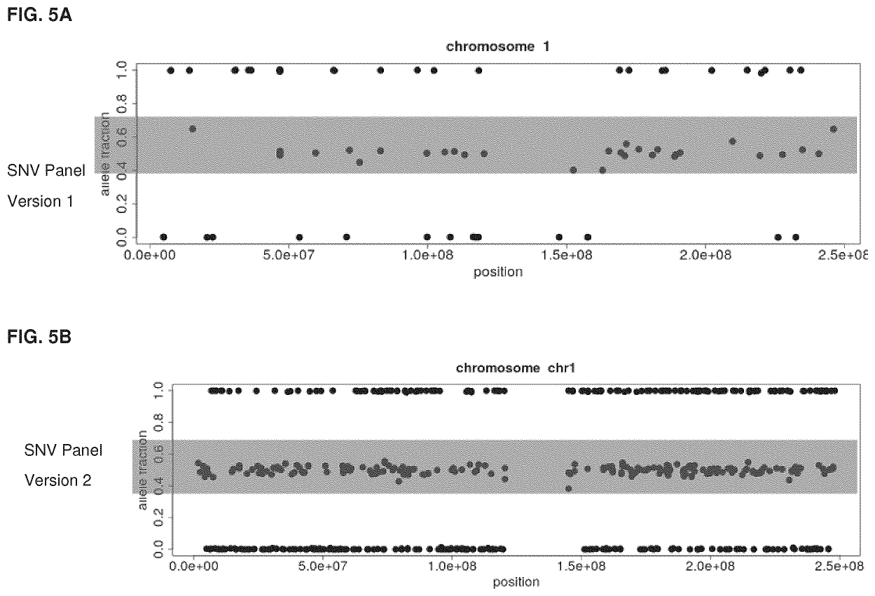

SNV Panel Version 2 was designed with increased SNV coverage. This panel has 5× increased density of heterozygous SNVs compared to SNV Panel Version 1 (See FIGS. 5A and 5B), and thus can provide more accurate identification of LOH, as evaluated against WGS.

The SNVs were selected for SNV Panel Version 2 according to the following selection criteria based on the population and sequencing characteristics.

The population characteristics were:
(1) 33%<allele frequency<66%, and
(2) 0<inbreeding coefficient<0.2.

The 5′-flanking sequence (50 base pairs) characteristics were:
(1) GC percentage between 25% and 75%,
(2) unique, and
(3) not containing other high frequency SNVs.

Additional primers were added for an additional four thousand (4000) population single nucleotide variants (SNVs) to provide additional coverage. In order to improve compatibility with low-quality FFPE input (multiple clinical sources), primer pairs in close proximity to each target are favored. In order to include SNVs which are useful for the detection of copy neutral loss of heterozygosity (LOH) structural variations, SNVs that are commonly heterozygous across sub-populations according to the current gnomAD release (v3) are selected. In order to select SNVs that are likely to yield the highest quality and quantity of NGS reads with AMP chemistry, SNVs in amplicons that are adjacent to repetitive or high GC regions of the genome were avoided, and SNVs that are less prone to noise introduced during PCR or sequencing (e.g., are not adjacent to polynucleotide tracts) are favored. Finally, in order to select SNVs that allow for spatial granularity in genomic calls, SNVs which are as evenly distributed throughout the genome as possible are selected.

Genomic DNA (>50 ng) was extracted from FFPE samples of multiple solid tumor types (n=43). Next-generation sequencing was performed on anchored multiplex PCR libraries, constructed using probes that incorporate unique molecular identifiers and span 26 genes and 5,000 genome-wide common germline SNVs. Unmatched non-tumor samples (n=24) were used to generate a reference baseline dataset. The FACETS algorithm, optimized to account for differential DNA fragmentation across samples, was used to assess copy number imbalance in heterozygous SNVs and to quantify tumor purity. Allele fractions at each heterozygous SNV were used to estimate allelic imbalances across chromosomal regions. A reference dataset was derived from matched FFPE tumor samples by whole genome sequencing (WGS) and analysis of sequence data using 3 complementary algorithms. Allele-specific copy number analysis and tumor purity estimation from SNV Panel Version 2 and WGS data were compared.

Copy number was evaluable in 605 genes from 24 matched tumor samples that passed quality control filters. Median sequencing depth across samples by SNV Panel Version 2 and WGS were 1346× and 18.6×, respectively. LOH detection by SNV Panel Version 2 was reproducible (100%) across 170 genes from 7 samples analyzed in duplicate. A strong correlation was observed between sample purity estimates by WGS and SNV Panel Version 2 (Pearson's r=0.81, p<0.001). Compared with WGS-derived calls, the sensitivity and specificity of LOH detection by SNV Panel Version 2 were 95% and 90%, respectively, rising to 97% and 91% in regions with LOH agreement by all 3 WGS algorithms, and to 99% and 97% in diploid regions with no subclonal alterations.

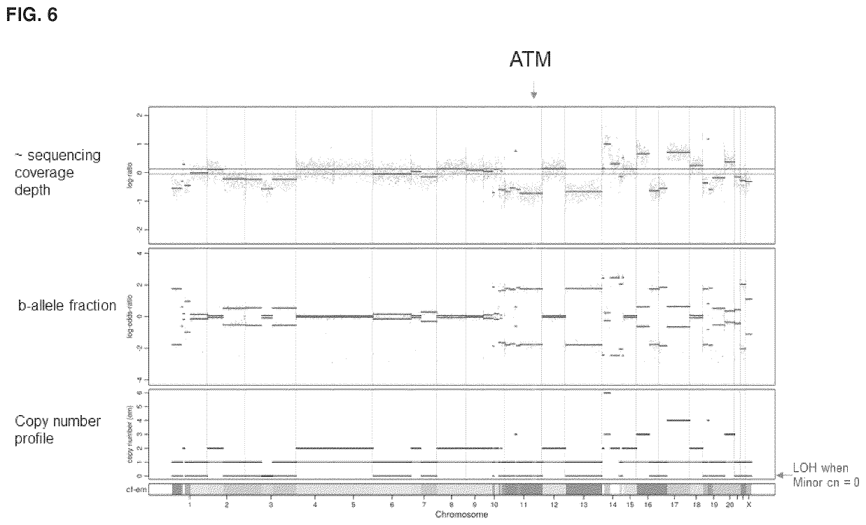

FIG. 6 demonstrates an example of copy number profile assembled using the SNV Panel Version 2 for a cancer cell having a biallelic ATM loss of function.

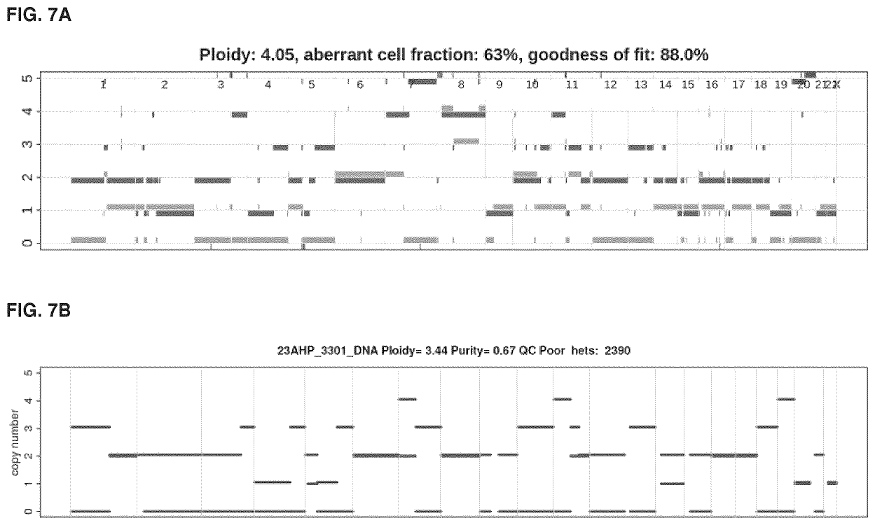

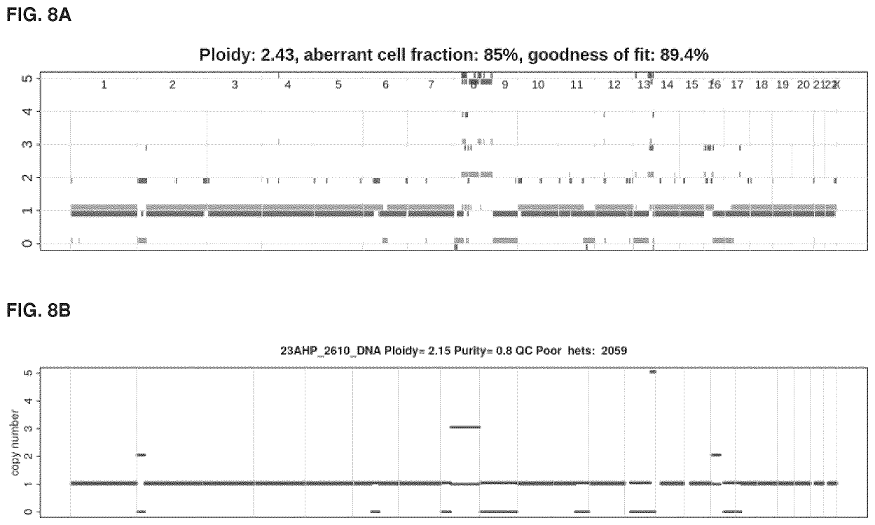

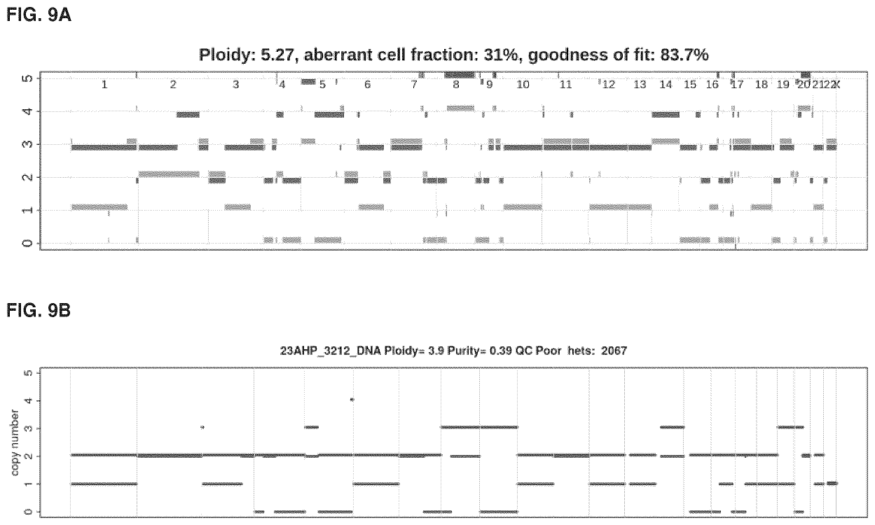

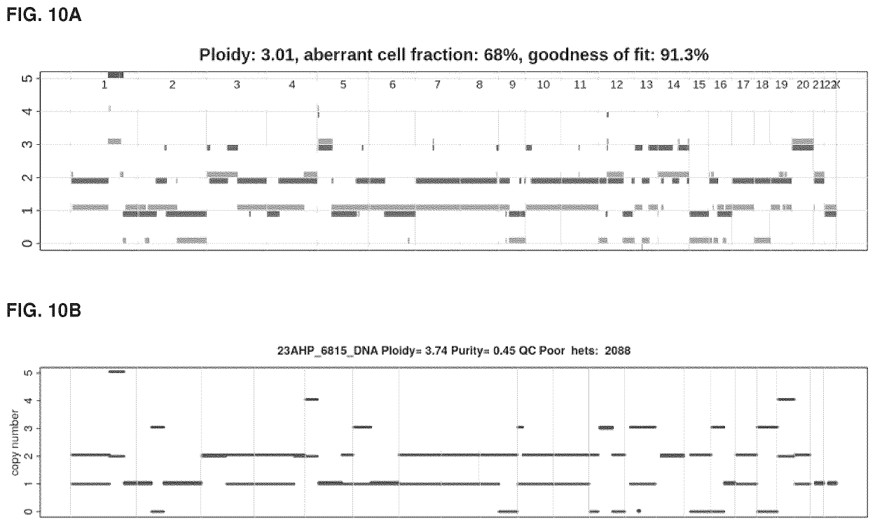

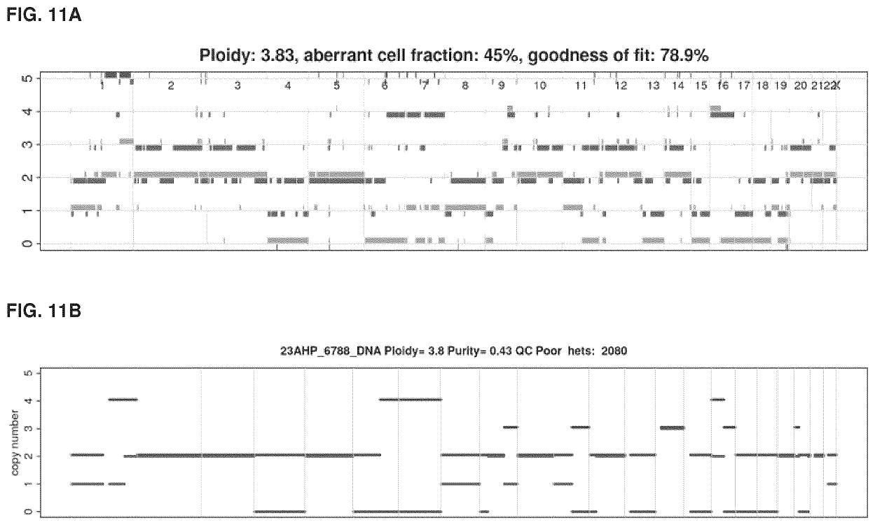

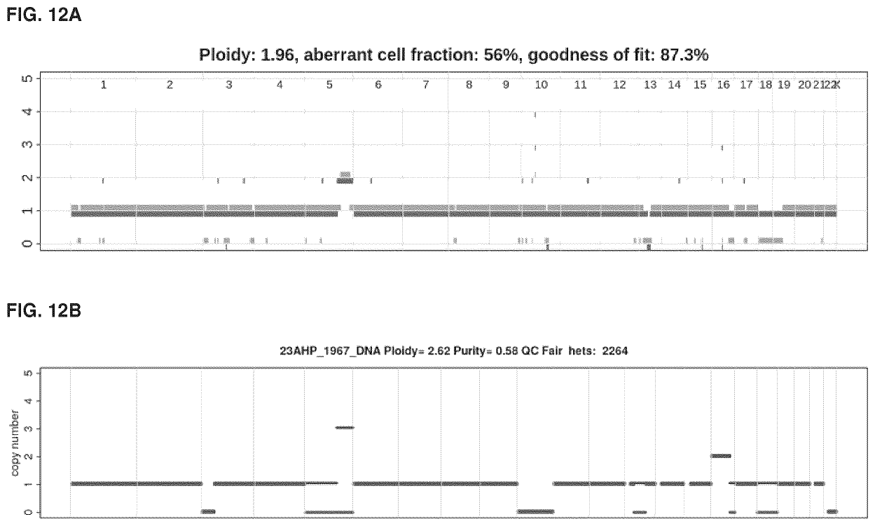

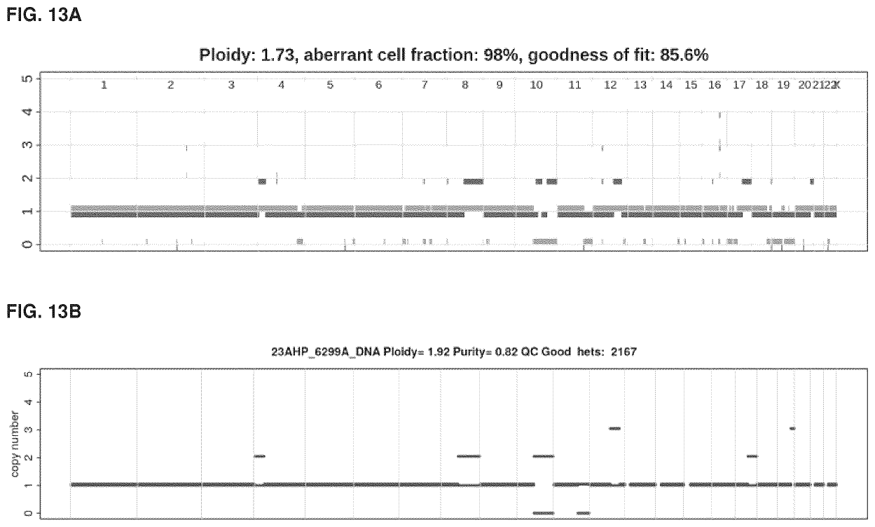

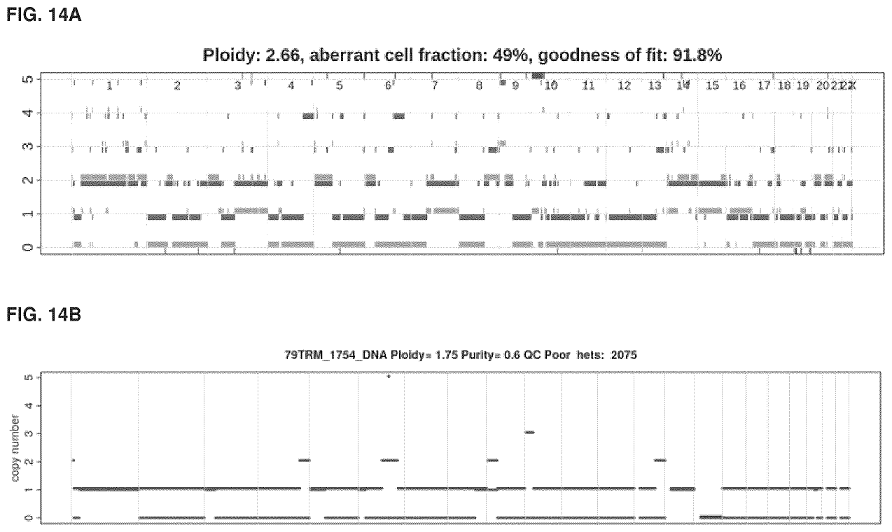

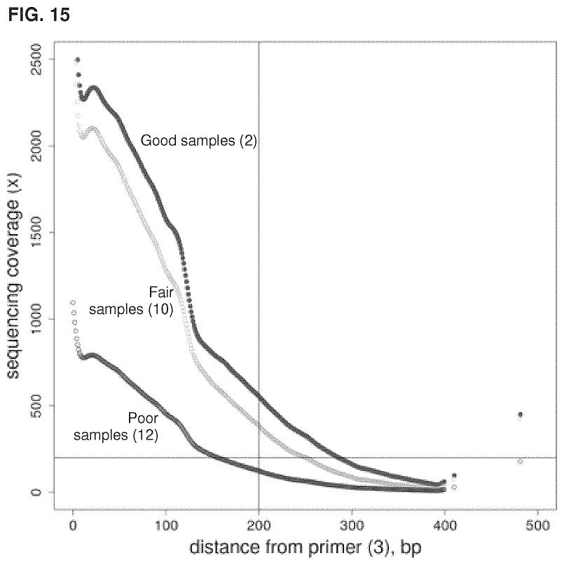

Quantification and comparison of copy number calls across all chromosomes as determined by WGS and the SNV Panel Version 2 for the 8 exemplary samples are shown in FIGS. 7A-14B. In particular, the ability for the SNV Panel Version 2 to accurately identify LOH was evaluated, with WGS serving as the basis for comparison. SNV Panel and WGS exhibited good agreement in calling LOH in the 8 exemplary samples (see Tables 5-12).

TABLE 5
Concordance of LOH calls per gene

WGS

| | | LOH | Heterozygosity |
|---|---|---|---|
| SNV Panel | LOH | 10 | 0 |
| Version 2 | Heterozygosity | 1 | 15 |

TABLE 6
Concordance of LOH calls per gene

WGS

| | | LOH | Heterozygosity |
|---|---|---|---|
| SNV Panel | LOH | 7 | 0 |
| Version 2 | Heterozygosity | 0 | 19 |

TABLE 7
Concordance of LOH calls per gene

WGS

| | | LOH | Heterozygosity |
|---|---|---|---|
| SNV Panel | LOH | 6 | 3 |
| Version 2 | Heterozygosity | 0 | 17 |

TABLE 8
Concordance of LOH calls per gene

WGS

| | | LOH | Heterozygosity |
|---|---|---|---|
| SNV Panel | LOH | 5 | 0 |
| Version 2 | Heterozygosity | 0 | 21 |

TABLE 9
Concordance of LOH calls per gene

WGS

| | | LOH | Heterozygosity |
|---|---|---|---|
| SNV Panel | LOH | 10 | 2 |
| Version 2 | Heterozygosity | 3 | 11 |

TABLE 10
Concordance of LOH calls per gene

WGS

| | | LOH | Heterozygosity |
|---|---|---|---|
| SNV Panel | LOH | 4 | 2 |
| Version 2 | Heterozygosity | 2 | 18 |

TABLE 11
Concordance of LOH calls per gene

WGS

| | | LOH | Heterozygosity |
|---|---|---|---|
| SNV Panel | LOH | 2 | 0 |
| Version 2 | Heterozygosity | 0 | 24 |

TABLE 12
Concordance of LOH calls per gene

WGS

| | | LOH | Heterozygosity |
|---|---|---|---|
| SNV Panel | LOH | 19 | 4 |
| Version 2 | Heterozygosity | 0 | 3 |

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A method of identifying a cell from a subject as having a biallelic mutation in a target gene, the method comprising:
from read counts for a plurality of consistently covered single nucleotide variants (SNVs) comprising homozygous and heterozygous consistently covered SNVs obtained from sequencing a sample comprising the cell and from reference read counts, determining an integer total copy number of a locus segment within a target gene region in the cell from the subject and/or two integer allele-specific copy numbers of the locus segment, the target gene region comprising the mutation, wherein the reference read counts are from a panel of normal samples,
wherein the cell is identified as having a biallelic mutation for a target gene,
if at least one of the integer total copy number and the integer allele-specific copy numbers is 0, provided that the remaining target gene allele, if present, comprises the mutation, or
if none of the integer allele-specific copy numbers is 0 and target gene alleles are present, each of the targe gene alleles independently having the mutation.

2. The method of claim 1, wherein the determining step comprises:
from the read counts and the reference read counts, determining total copy number log-ratios, allelic copy number log-odds ratios, and target coverage values for the SNVs;
segmenting the total copy number log-ratios and the allelic copy number log-odds ratios;
estimating sample purity and sample ploidy for the cell from the total copy number log-ratios and the target coverage values; and
from the target coverage values, the sample purity, the sample ploidy, the total copy number log-ratios, and the allelic copy number log-odds ratios, generating an integer total copy number of a segment comprising a plurality of SNVs within a target gene region in the cell and two integer allele-specific copy numbers of the segment.

3. The method of claim 2, wherein the method further comprises adjusting the ratios for location shift.

4. A method of identifying a target mutation in a cell from a subject as being germline or somatic, the method comprising:
from read counts for a plurality of consistently covered single nucleotide variants (SNVs) comprising homozygous and heterozygous consistently covered SNVs obtained from sequencing a sample comprising the cell and from reference read counts, determining an observed allele fraction of a locus segment within a target gene region in the cell from the subject, the target gene region comprising the target mutation;
determining expected allele fractions for a germline target mutation and for a somatic target mutation;
comparing the observed allele fraction to the expected allele fractions to identify the most probable of the germline and somatic mutations; and
identifying the target mutation as germline or somatic as that which is the most probable for the germline and somatic mutations.

5. The method of claim 4, wherein the cell is in a sample from the subject, and the sample is impure (Φ<0.9).

6. The method of claim 4 or 5, wherein the comparing step is performed using Bayesian model comparison.

7. The method of any one of claims 1 to 6, wherein each of the consistently covered SNVs has the mean coverage of at least 200× across reference non-cancerous samples.

8. The method of any one of claims 1 to 7, wherein the plurality of SNVs comprises frequent SNVs, the frequent SNVs having an allele frequency of 33% to 66% in humans.

9. The method of claim 8, wherein the plurality of SNVs comprises SNVs disposed at most 300 base pairs away from the frequent SNVs.

10. The method of any one of claims 1 to 6, wherein the plurality of SNVs comprises SNVs, each of the SNVs having a 5′-flanking sequence of at least 20 contiguous nucleobases comprising 25-75% GC content, wherein the 5′-flanking sequence is unique and does not comprise other SNVs.

11. The method of any one of claims 1 to 10, wherein the plurality of SNVs comprises at least 20 heterozygous SNVs.

12. The method of any one of claims 1 to 11, wherein the target gene region comprises the target gene and flanking regions up to 10 kilobases each.

13. The method of any one of claims 1 to 11, wherein the target gene region comprises the target gene and flanking regions up to 5 kilobases each.

14. The method of any one of claims 1 to 11, wherein the target gene region comprises the target gene and flanking regions up to 2 kilobases each.

15. The method of any one of claims 1 to 14, wherein the target gene region is a target exome region.

16. The method of any one of claims 1 to 14, wherein the target gene region is a target transcriptome region.

17. The method of any one of claims 1 to 14, wherein the target gene region is a target genome region.

18. A method of identifying a target mutation in a cell from a subject as being germline or somatic, the method comprising identifying the target mutation in the normal, matched sample from the subject,
wherein
if the target mutation present in the cell from the subject is identified in the normal, matched sample, the target mutation is germline, and
if the target mutation present in the cell from the subject is not identified in the normal, matched sample, the target mutation is somatic.

19. The method of any one of claims 1 to 18, wherein the cell from the subject is a cancer cell from the subject.

20. The method of any one of claims 1 to 19, wherein the target is STAG2.

21. The method of any one of claims 1 to 19, wherein the target is SETD2.

22. The method of any one of claims 1 to 19, wherein the target is CDK12.

23. The method of any one of claims 1 to 19, wherein the target is ATRIP.

24. The method of any one of claims 1 to 19, wherein the target is REV3L.

25. The method of any one of claims 1 to 19, wherein the target is RAD17.

26. The method of any one of claims 1 to 19, wherein the target is CHTF8.

27. The method of any one of claims 1 to 19, wherein the target is FZR1.

28. The method of any one of claims 1 to 19, wherein the target is RAD51B.

29. The method of any one of claims 1 to 19, wherein the target is RAD51C.

30. The method of any one of claims 1 to 19, wherein the target is RAD51 D.

31. The method of any one of claims 1 to 19, wherein the target is PALB2.

32. The method of any one of claims 1 to 19, wherein the target is RNASEH2A.

33. The method of any one of claims 1 to 19, wherein the target is RNASEH2B.

34. The method of any one of claims 1 to 33, wherein the mutation is a germline mutation.
